(19) United States
(12) Patent Application Publication
(10) Pub. No.: US 2016/0143749 A1
(43) Pub. Date: May 26, 2016

(54) GLENOID COMPONENT INSTALLATION PROCEDURE AND TOOLING FOR SHOULDER ARTHROPLASTY

(71) Applicants: Aaron B. PANONE, Sommerville, MA (US); Michael WOLLOWITZ, Chatham, NY (US); Arun SHANBHAG, Arlington, MA (US)

(72) Inventors: Thomas F. HOLOVACS, Hingham, MA (US); Aaron B. PANONE, Sommerville, MA (US); Michael WOLLOWITZ, Chatham, NY (US); Arun SHANBHAG, Arlington, MA (US)

(73) Assignee: THE GENERAL HOSPITAL CORPORATION, Boston, MA (US)

(21) Appl. No.: 14/110,225

(22) PCT Filed: Apr. 6, 2012
(86) PCT No.: PCT/US2012/032518

§ 371 Date: May 15, 2014

Related U.S. Application Data
(60) Provisional application No. 61/473,520, filed on Apr. 8, 2011.

(51) Int. Cl. A61F 2/46; A61B 17/16; A61F 2/40

(52) U.S. Cl. CPC A61F 2/4612; A61F 2/4081; A61B 17/1684; A61B 2017/1778

(57) ABSTRACT

A method for implanting a glenoid component during shoulder arthroplasty is disclosed. In the method, a template is placed against a surface of a glenoid cavity of a scapula, and a bone marking tool is inserted through a cut-out guide in the template. The bone marking tool is used to create a bone mark on the surface of the glenoid cavity. A drill is aligned with the bone mark and a first pilot hole is drilled in the surface of the glenoid cavity. A locating pin of a first drill jig is placed in the first pilot hole, and a drill is inserted through a guide hole of the first drill jig to drill a second pilot hole in the surface of the glenoid cavity. Bone material is removed between the first pilot hole and the second pilot hole to create a slot in the surface of the glenoid cavity between the first pilot hole and the second pilot hole. A keel of a glenoid component is positioned in the slot, and the glenoid component is secured in the scapula. A kit is also provided for use with the method. The kit includes a glenoid component including a body having a first articulating surface and a second surface opposite the first articulating surface. The first articulating surface is dimensioned for engaging a head of a humerus or a humeral implant. The second surface is dimensioned for being secured to a scapula. The glenoid component further includes a keel extending away from the second surface. The kit also includes a transparent template having a cut-out guide dimensioned to receive a bone marking tool. The template preferably has an indication of a centerline of the template. The kit also includes a first drill jig having a locating pin and a guide hole dimensioned to receive a drill bit, and a cutting tool having a follower pin and a cutting surface.

### CROSS-REFERENCES TO RELATED APPLICATIONS

This application claims priority from U.S. Patent Application No. 61/473,520 filed Apr. 8, 2011.

### STATEMENT REGARDING FEDERALLY SPONSORED RESEARCH

Not Applicable.

### BACKGROUND OF THE INVENTION

1. Field of the Invention

This invention relates to a suite of custom tooling and a surgical technique for a process in which a prosthetic glenoid component is inset into the face of the existing glenoid bone.

2. Description of the Related Art

Total shoulder replacement is a well-accepted treatment option for patients with osteoarthritis of the shoulder (glenohumeral joint). This procedure involves replacement of both the humeral head and the glenoid. The humeral head can be replaced/resurfaced with either a stemmed implant which is secured, in part, in the canal of the humerus or a stemless implant which is secured with the humeral head itself. The glenoid is a pear-shaped bone which is part of the scapula. It is the “socket” of the shoulder. The bony geometry of the glenoid is such that there is a shallow concavity which comprises the socket. The glenoid is small (approximately 3 cm.×4 cm. dimension). Glenoid replacement/resurfacing is accomplished by initially preparing the glenoid by performing a circumferential reaming of a constant diameter. The reaming prepares the glenoid by removing any remaining cartilage and bone such that the subchondral bone is exposed. The cortical bone of the glenoid, which outlines the peripheral shape of the glenoid is reamed along with the rest of the glenoid surface. Once reaming and preparation of the glenoid is accomplished, a glenoid-shaped implant is cemented onto the glenoid to serve as the bearing surface for the humeral articulation with the glenoid.

FIG. 1 shows an anterior view of a prior art right shoulder replacement. A glenoid component 12 is positioned in a resected portion of the glenoid cavity 13 of the scapula 14. The glenoid cavity 13 is located on the head of the scapula 14 between the external border 15 and upper border 16, and between the acromion process 17 and coracoid process 18. The clavicle 19 is positioned above the glenoid component 12. The humeral prosthesis 21 is attached to the proximal end of the humerus 22. A spherical head 23 of the humeral prosthesis 21 articulates with the glenoid component 12 which extends outward from the natural bearing surface of the glenoid cavity 13.

Loosening of glenoid components can be a problem in total shoulder arthroplasty. The consequence of glenoid component loosening can be severe, as a loosened glenoid component cannot always be replaced because of bone deficiencies. Therefore, there is a need for surgical procedures and associated tooling that improve the fixation of glenoid components and that preserve the cortical region of bone in the event a revision procedure is required.

### SUMMARY OF THE INVENTION

The present invention provides a suite of custom tooling and a surgical technique for a process in which a prosthetic glenoid component is inset into the face of the existing glenoid bone. The tooling and a surgical technique preserve the cortical region of bone and reduce the chances of delamination of the prosthetic glenoid component from the glenoid. The invention includes a set of reduced-size prosthetic glenoid inserts, and corresponding sized tooling. Advantageous tooling features include a cutting device which is capable of a plunge and lateral cut, which creates a concave recess allowing the prosthetic insert to sit flush with the surface of the preserved ring of cortical glenoid bone. The path of the cutting tool is restricted by a smaller diameter follower pin which traces the inside perimeter of a full-radius slot which is cut into the glenoid prior to the recess cutting operation. Sized prosthetic glenoid inserts, the tooling suite, and documentation or training on the associated surgical procedure can be packaged as a single product.

Reaming only the bearing part of the glenoid and leaving the cortex intact allows the glenoid component to be seated “into” the bone of the glenoid as opposed to “onto” it. This would impart stability to the glenoid component and, consequently, longevity of the implant. Once reaming and preparation is performed, the glenoid component can be implanted “into” the glenoid in a secure and stable manner.

In one aspect, the invention provides a kit for use with a method for implanting a glenoid component during shoulder arthroplasty. The kit includes a glenoid component including a body having a first articulating surface and a second surface opposite the first articulating surface. The first articulating surface is dimensioned for engaging a head of a humerus or a humeral implant. The second surface is dimensioned for being secured to a scapula. The glenoid component further includes a keel extending away from the second surface. The kit also includes a transparent template having a cut-out guide dimensioned to receive a bone marking tool. The template preferably has an indication of a centerline of the template. The kit also includes a first drill jig having a locating pin and a guide hole dimensioned to receive a drill bit, and a cutting tool having a follower pin and a cutting surface. The cutting surface of the cutting tool can be dimensioned to create a recess in the surface of the glenoid cavity of the scapula wherein the recess has a size such that a periphery of the glenoid bone is preserved when the recess is created in the surface of the glenoid cavity.

In one form, the template includes an outline of a footprint of the glenoid component. The cutting tool can include a depth-stop collar for adjusting depth of cut of the cutting tool, and the cutting surface of the cutting tool can have a first curvature approximately equal to a second curvature of the second surface of the glenoid component. The first drill jig can include an indication of a centerline of the first drill jig on a top surface of the first drill jig.

The kit can further include a second drill jig having (i) a locating plug, (ii) a first guide bore dimensioned to receive a drill bit, and (iii) a second guide bore dimensioned to receive the drill bit. The second drill jig can include an indication of a centerline of the second drill jig on a top surface of the second drill jig. The locating plug of the second drill jig can be oblong.

The kit can further include a drill bit dimensioned to pass through the guide hole of the first drill jig. The kit can further include a drill stop for adjusting depth of cut of the drill bit.

In one form, the keel of the glenoid component is oblong. The follower pin and the cutting surface of the cutting tool can be concentric and integral. The cutting tool can include a shank dimensioned to be secured in a chuck of a surgical power drill.

In one form, an outer perimeter of the first drill jig has the same geometry as an outer edge of the glenoid component.

The kit can further include a plurality of the glenoid component, and at least two of the plurality of the glenoid component can have a different size. The kit can further include a plurality of the template, and at least two of the plurality of the template can have an outline of a footprint of two different sized glenoid components.

In one form, the body of the glenoid component is dimensioned such that the glenoid component is flush with native peripheral glenoid bone when the glenoid component is secured to the scapula.

In another aspect, the invention provides a method for implanting a glenoid component during shoulder arthroplasty. In the method, a template is placed against a surface of a glenoid cavity of a scapula, and a bone marking tool is inserted through a cut-out guide in the template. The bone marking tool is used to create a bone mark on the surface of the glenoid cavity. A drill is aligned with the bone mark and a first pilot hole is drilled in the surface of the glenoid cavity. A locating pin of a first drill jig is placed in the first pilot hole, and a drill is inserted through a guide hole of the first drill jig to drill a second pilot hole in the surface of the glenoid cavity. Bone material is removed between the first pilot hole and the second pilot hole to create a slot in the surface of the glenoid cavity between the first pilot hole and the second pilot hole. A keel of a glenoid component is positioned in the slot, and the glenoid component is secured in the scapula. The slot and the keel can be oblong.

In the method, the step of placing the template against a surface of a glenoid cavity of a scapula can include marking a centerline on the surface of the glenoid cavity of the scapula and aligning a line on the template with the centerline when placing the template against the surface of the glenoid cavity of the scapula. The template can be transparent, and the template can include an outline of a footprint of the glenoid component.

In the method, the step of removing bone material between the first pilot hole and the second pilot hole to create a slot in the surface of the glenoid cavity between the first pilot hole and the second pilot hole can include placing a follower pin of a cutting tool in the slot and moving the follower pin from a first position to a second position in the slot such that a cutting surface of the cutting tool removes bone material thereby creating a recess in the surface of the glenoid cavity surrounding the slot. The step of positioning the keel of the glenoid component in the slot and securing the glenoid component in the scapula can include positioning a body of the glenoid component in the recess.

In one form, the cutting tool includes a depth-stop collar for adjusting depth of cut of the cutting tool. The depth-stop collar can be secured in a position such that a length of exposed cutting tool is approximately the same as the thickness of the glenoid component.

In the method, the step of removing bone material between the first pilot hole and the second pilot hole to create a slot in the surface of the glenoid cavity between the first pilot hole and the second pilot hole can include placing a locating plug of a second drill jig in the slot, and drilling through a first guide bore of the second drill jig to lengthen one end of the slot, and drilling through a second guide bore of the second drill jig to lengthen an opposite end of the slot.

In one form, the recess and the body of the glenoid component are oblong. The cutting surface of the cutting tool can have a first curvature approximately equal to a second curvature of a surface of the glenoid component that contacts the surface of the glenoid cavity when the glenoid component is secured in the scapula.

In the method, the step of placing the template against a surface of a glenoid cavity of a scapula can include marking a centerline on the surface of the glenoid cavity of the scapula, and the step of placing the locating pin of the first drill jig in the first pilot hole can include aligning a line on the first drill jig with the centerline.

In the method, the step of aligning the drill with the bone mark and drilling a first pilot hole in the surface of the glenoid cavity can include drilling the first pilot hole in the surface of the glenoid cavity by advancing a drill bit into the surface of the glenoid cavity until a drill stop on the drill bit contacts the surface of the glenoid cavity.

In the method, the step of drilling through the guide hole of the first drill jig to create the second pilot hole in the surface of the glenoid cavity can include creating the second pilot hole in the surface of the glenoid cavity by advancing a drill bit through the guide hole of the first drill jig until a drill stop on the drill bit contacts a surface of the first drill jig.

In the method, the step of positioning the keel of a glenoid component in the slot and securing the glenoid component in the scapula can include securing the glenoid component in the scapula such that a bearing surface of the glenoid component lines up with a non-resected section of the surface of the glenoid cavity.

An outer perimeter of the first drill jig can have the same geometry as an outer edge of the glenoid component. The method can be used with a robotic orthopedic surgical system.

In the method, the step of removing bone material between the first pilot hole and the second pilot hole to create a slot in the surface of the glenoid cavity between the first pilot hole and the second pilot hole can include placing a follower pin of a cutting tool in the slot and moving the follower pin from a first position to a second position in the slot such that a cutting surface of the cutting tool removes bone material thereby creating a recess in the surface of the glenoid cavity surrounding the slot wherein the cutting surface of the cutting tool is dimensioned such that the recess created has a size such that a periphery of the glenoid bone is preserved. The step of positioning the keel of the glenoid component in the slot and securing the glenoid component in the scapula can include positioning a body of the glenoid component in the recess such that the glenoid component is flush with native peripheral glenoid bone.

Thus, the invention can be used in total shoulder arthroplasty which involves replacement of the humeral head and glenoid articulating surfaces. The glenoid is one focus of this invention, technique and implant design. Glenoid arthroplasty involves “replacement” of articular cartilage (which is absent in arthritis) with a prosthetic device. Previously, this involved initially preparing the glenoid surface by circumferentially reaming the entire glenoid surface to achieve a uniform surface so that the implant can be placed on top of the native bone. The problems with the on top placement on the glenoid bone is solved with the technique/implant of the invention.

Previous onlay devices resulted in edge-loading of the prosthesis by the corresponding humeral head as it articulates. This can cause loosening and subsequent failure. The present technique/implant is an “inset” device—the periphery of the glenoid bone is preserved and only the inside of the bone is removed so that the implant can be placed flush with the native peripheral bone—i.e., not on top of it. The inset design will be dramatically more stable and, therefore, more durable.

Furthermore, the preparation of the glenoid for insertion of the inset glenoid component will also be dramatically different (easier) than current techniques. The preparation of the glenoid for current onlay device is technically challenging—the circumferential reamer is necessarily large so that the entire glenoid bone surface can be removed. The tooling/implant of the invention will dramatically simplify the preparation of the glenoid because a significantly smaller area of bone needs to be removed.

The custom tooling of the invention includes a cutting tool which follows a pilot slot and cuts out an inset in the glenoid in one step. A working template is used to determine appropriate device size and fit for the glenoid. In the method, there is provided a technique to drill pilot holes and cut the slot. The cutting tool is used with a follower pin to cut the inset out with appropriate depth controls. There are tooling options to cut out an oval or pear shaped concavity into the glenoid. While a flat bottomed cut is acceptable, it is preferred to use a convex cutting tool to create a concave (rather than flat) bottom for insertion of a dished implant.

The custom tooling of the invention includes a transparent template. The surgeon places this template on the exposed glenoid surface and aligns cross markings which the surgeon has marked on the glenoid with the center on this template. The surgeon can now determine how much border/collar of cortical bone to retain and thus what size of inset glenoid component to implant.

These and other features, aspects, and advantages of the present invention will become better understood upon consideration of the following detailed description, drawings and appended claims.

### BRIEF DESCRIPTION OF THE DRAWINGS

FIG. 1 shows an anterior view of a prior art right shoulder replacement.

FIG. 2 is a partial lateral view of the scapula showing the glenoid cavity with centerline markings from a bone marking tool.

FIG. 3 is a partial lateral view of the scapula showing the glenoid cavity with a transparent marking template placed on the surface of the glenoid cavity.

FIG. 4 shows side elevational views of a drill bit with a drill-stop collar adjusted to three different positions, the drill bit and collar being suitable for use during the prosthetic glenoid installation procedure of the invention.

FIG. 5A shows a side elevational view, partially in cross-section, of a first drill jig and drill bit and collar suitable for use during the prosthetic glenoid installation procedure of the invention.

FIG. 5B shows a top plan view of the first drill jig of FIG. 5A.

FIG. 6 is a partial lateral view of the scapula showing the glenoid cavity with drilled holes and centerline markings.

FIG. 7 is a partial lateral view of the scapula showing the glenoid cavity with an oblong slot and centerline markings.

FIG. 8 shows a side elevational view of a follower-bore cutting tool and a depth-stop collar, the follower-bore cutting tool and collar being suitable for use during the prosthetic glenoid installation procedure of the invention.

FIG. 9A is a partial lateral view of the scapula showing the glenoid cavity with an oblong slot surrounded by a larger concave oblong recess.

FIG. 9B is a partial cross-sectional view taken along line 9B-9B of FIG. 9A showing the oblong slot surrounded by a larger concave oblong recess in the glenoid cavity.

FIG. 9C is a partial cross-sectional view taken along line 9C-9C of FIG. 9A showing the oblong slot surrounded by a larger concave oblong recess in the glenoid cavity.

FIG. 10A shows a side elevational view, partially in cross-section, of a second drill jig and drill bit and collar suitable for use during the prosthetic glenoid installation procedure of the invention.

FIG. 10B shows a top plan view of the second drill jig of FIG. 10A.

FIG. 11 is a partial lateral view of the scapula showing the glenoid cavity with an oblong slot surrounded by a larger concave oblong recess.

FIG. 12 is a partial cross-sectional view taken along line 12-12 of FIG. 11 showing a glenoid component implanted in a glenoid cavity partially resected using the prosthetic glenoid installation procedure of the invention.

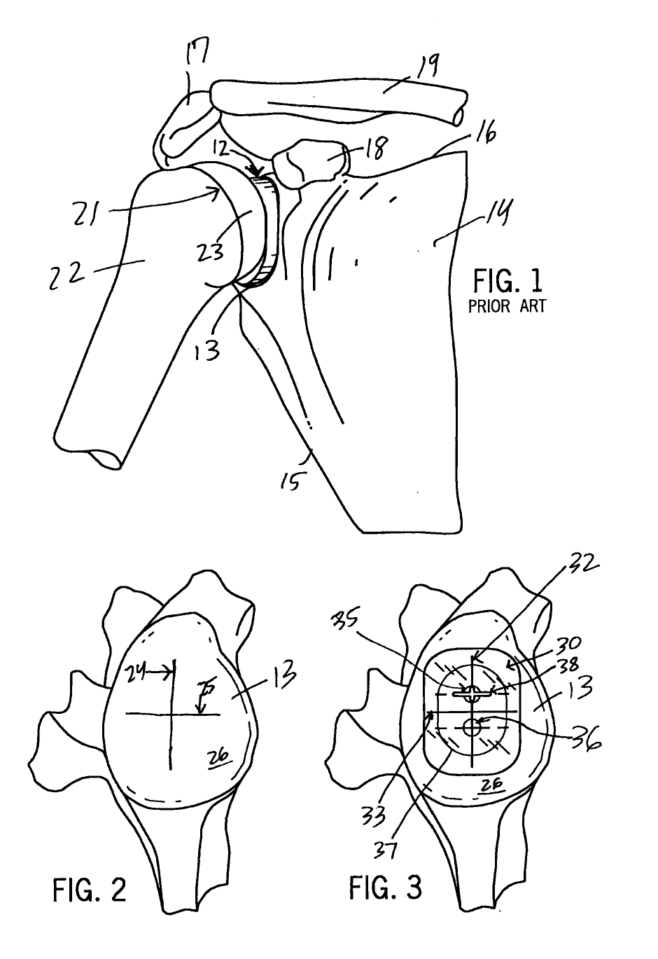

Like reference numerals will be used to refer to like parts from Figure to Figure in the following description of the drawings.

### DETAILED DESCRIPTION OF THE INVENTION

One non-limiting example version of a glenoid component installation procedure and tooling according to the invention is shown in FIGS. 2 to 12. The tooling and glenoid component can be provided as a kit for use with the method of the invention.

First, the glenoid surface is prepared. The surgeon marks an approximate vertical (superior to inferior) centerline 24 and a horizontal (anterior to posterior) centerline 25 on the surface 26 of the glenoid cavity 13 with a bone marking tool such as a cauterizer. See FIG. 2.

The appropriate size of glenoid component is selected for the application, and a corresponding size of transparent template 30 is placed over the surface 26 of the glenoid cavity 13 as shown in FIG. 3. A template vertical centerline 32 and a horizontal centerline 33 on the template 30 are then aligned with the centerlines 24, 25 drawn on the glenoid surface. The surface of the template 30 includes a first printed indicia 35 showing approximate location of a first pilot hole, a second printed indicia 36 showing approximate location of a second pilot hole, and a third printed indicia 37 showing approximate location of the footprint of the appropriately sized prosthetic glenoid component. The glenoid component and the third printed indicia 37 can be matched in various sizes such as small, medium, large and extra large. A cross-shaped cut-out 38 in the superior-trabecular region of the template is used to guide a marking tool, such as a cauterizer, when indicating the position of a first pilot hole on the glenoid surface 26. The template 30 is then removed from the glenoid.

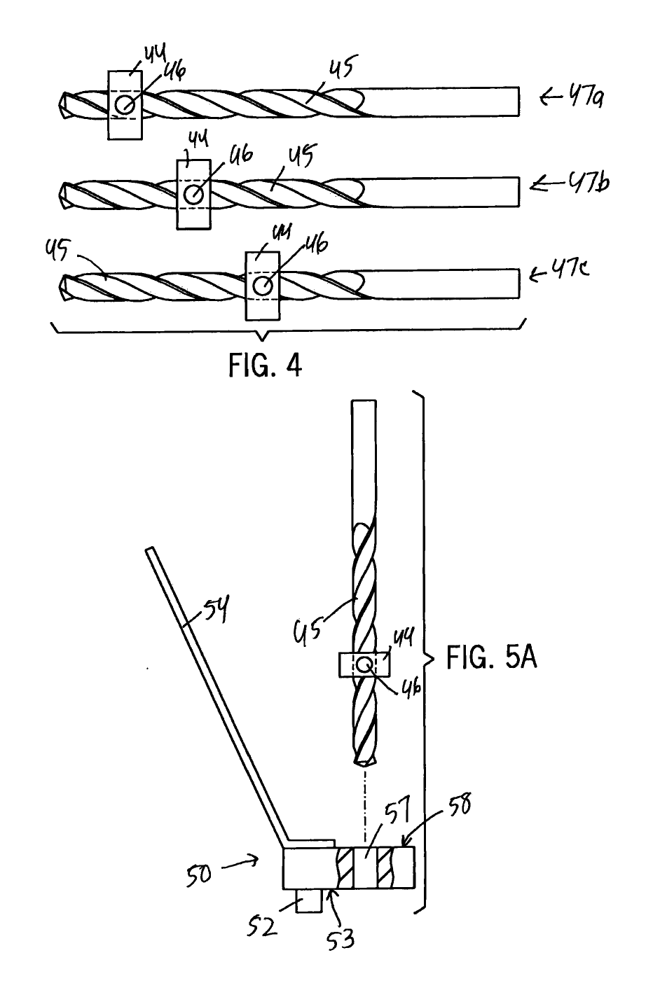

A first pilot hole 40 (shown in FIG. 6) is then drilled in the location marked in the previous step. FIG. 4 shows how a drill-stop collar 44 can be adjusted to three different depths during the prosthetic glenoid installation procedure. The drill bit 45 is marked with three different lines indicating the three different positions on the drill bit to help line up the drill-stop collar 44 on the drill bit 45. A set screw 46 can secure the drill-stop collar 44 on the drill bit 45. The drill-stop collar 44 on the drill bit 45 sets the depth of penetration of the drill bit 45 in the glenoid 13. For the first pilot hole drilling operation, the drill-stop collar 44 is set to the shallowest of the three positions 47a (shown at the top of FIG. 4).

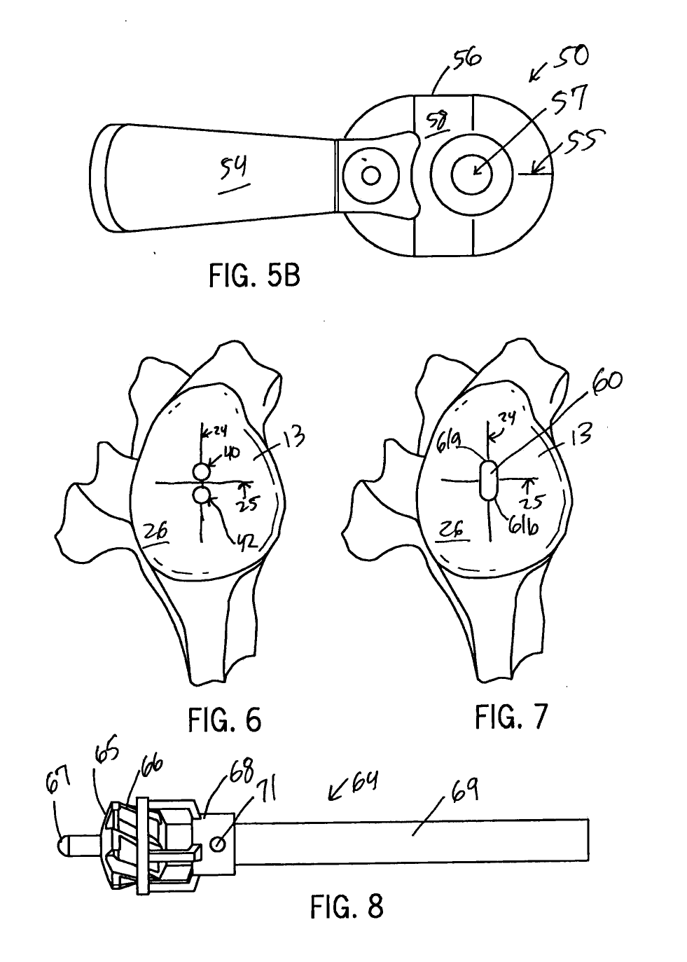

A second hole 42 is then drilled using a first drill jig 50 shown in FIGS. 5A and 5B. A cylindrical locating pin 52 extending from a bottom surface 53 of the first drill jig 50 is inserted into the first pilot hole 40 (shown in FIG. 6). A handle 54 is used for steadying the first drill jig 50, and the angle is set by the surgeon. A superior-inferior running line 55 on the first drill jig 50 is aligned with the vertical superior-inferior centerline 24 marked on the surface 26 of the glenoid 13. This sets the anterior-posterior swing angle of the first drill jig 50, and subsequent installation of the prosthetic glenoid. The outer perimeter 56 of the first drill jig 50 preferably has the same geometry as the outer edge of the prosthetic glenoid component that is going to be installed. This is to assist in the angular alignment and relative hole positioning.

Once the first drill jig 50 is oriented properly, the second hole 42 shown in FIG. 6 is drilled. For this operation, the drill-stop collar 44 on the drill bit 45 is set to the deepest of the three positions 47c in FIG. 4, since it has to travel through the first drill jig 50 to penetrate the surface of 26 the glenoid 13. The drill bit 45 is inserted into a guiding hole 57 on the first drill jig 50 and pressure is applied until the drill stop collar 44 touches the top surface 58 of the first drill jig 50, indicating that the second hole 42 is drilled to the proper depth.

After the second hole 42 is drilled, a chisel is used to remove the bone between the first hole 40 and the second hole 42, thereby making an oblong slot 60 with rounded ends 61a, 61b. The resulting slot 60 (shown in FIG. 7) in the glenoid surface 26 is approximately seven millimeters wide and sixteen millimeters long. This slot 60 is not yet long enough to fit the keel of the prosthetic glenoid insert. The slot 60 will be lengthened in a later step. The purpose of this slot 60 is to guide a cutting tool, which will create a recess so that the prosthetic glenoid insert can sit below the surface of the glenoid 13, and inside of the cortical region. The slot 60 has a depth of the keel of the prosthetic glenoid component to be installed.

FIG. 8 shows a follower-bore cutting tool 64 used in the method of the invention. The follower-bore cutting tool 64 features a convex cutting surface 65 on the bottom and cutting teeth 66 on the outer diameter as well, to allow for both plunge and lateral cutting. The cutting surface 65 has a matched curvature to that of the prosthetic glenoid component. In one non-limiting version, the cutting surface 65 has a diameter of about one inch. The cutting surface 65 is dimensioned to create a recess in the surface of the glenoid cavity wherein the recess has a size such that a periphery of the glenoid bone is preserved after cutting. The follower-bore cutting tool 64 includes a follower pin 67 at the distal end. The follower-bore cutting tool 64 uses slot 60 (see FIG. 7) as the pattern which is followed by the follower pin 67. The follower pin 67 limits the lateral travel of the cut path. In one non-limiting version, the follower pin 67 has an axial length of about 0.5 inches.

The depth of cut on the follower-bore cutting tool 64 is adjusted by setting the position of a depth-stop collar 68 which located on the shank 69 of the follower-bore cutting tool 64. The depth-stop collar 68 is secured in a position by a set screw 71 such that the length of exposed cutting tool is roughly the same as the thickness of the prosthetic glenoid component which is to be installed. Using the follower-bore cutting tool 64 at this setting should remove enough glenoid bone material to implant the prosthetic glenoid component into the glenoid surface so that the surface of the prosthetic glenoid component that is in contact with the humeral head is exactly where the original non-resected surface 26 of the glenoid 13 existed. The cutting surface 65 has a matched curvature to that of the prosthetic glenoid component.

The shank 69 of the follower-bore cutting tool 64 is secured in the chuck of a surgical power drill, and the smooth follower pin 67 of the follower-bore cutting tool 64 is placed into the superior end 61a of the slot 60 cut into the glenoid surface 26. Power is then supplied to the follower-bore cutting tool 64, and pressure is applied to the follower-bore cutting tool 64 until the follower-bore cutting tool 64 has plunged into the bone to the point where the depth-stop 68 has made contact with the cortical and existing surrounding trabecular bone. Lateral force is then applied to the follower-bore cutting tool 64, in the direction of the inferior trabecular region of the glenoid. Lateral force is applied and the follower-bore cutting tool 64 continues to remove bone until it has reached the inferior end 61b of the slot 60. The follower-bore cutting tool 64 is then powered off.

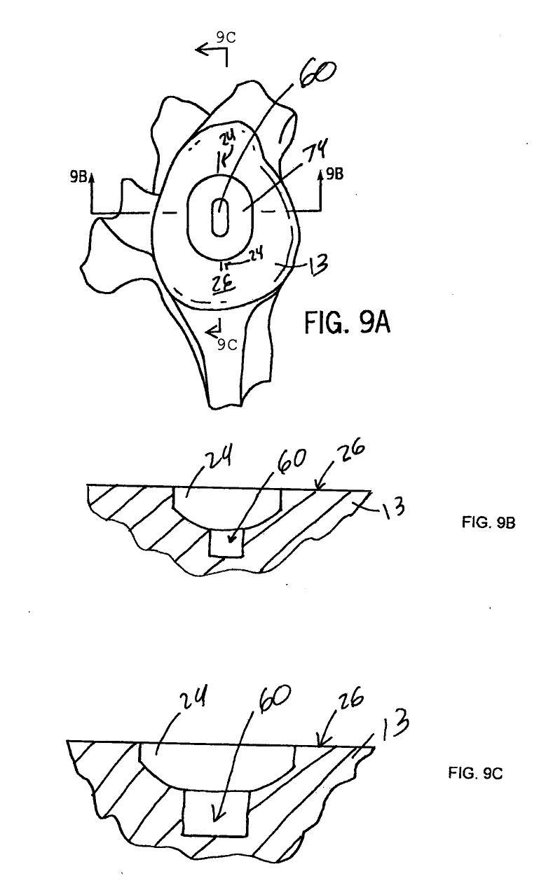

Looking at FIGS. 9A and 9B, at this point there exists the remaining depth of the slot 60 in the glenoid 13 surrounded by a much larger diameter concave slotted oblong recess 74, measuring approximately twenty-six millimeters wide by thirty-five millimeters long by seven millimeters deep. The concave slotted oblong recess 74 could also be fifteen to forty millimeters wide by twenty to fifty millimeters long by five to twenty millimeters deep.

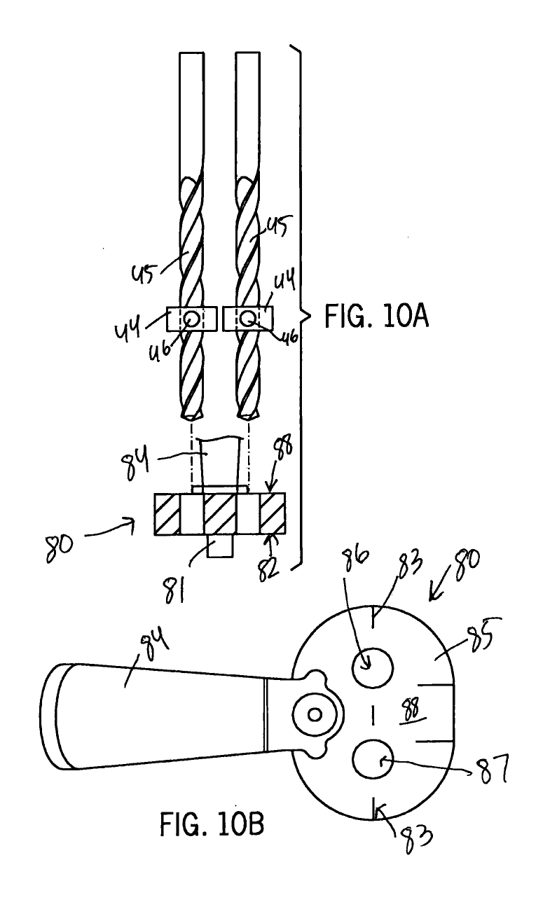

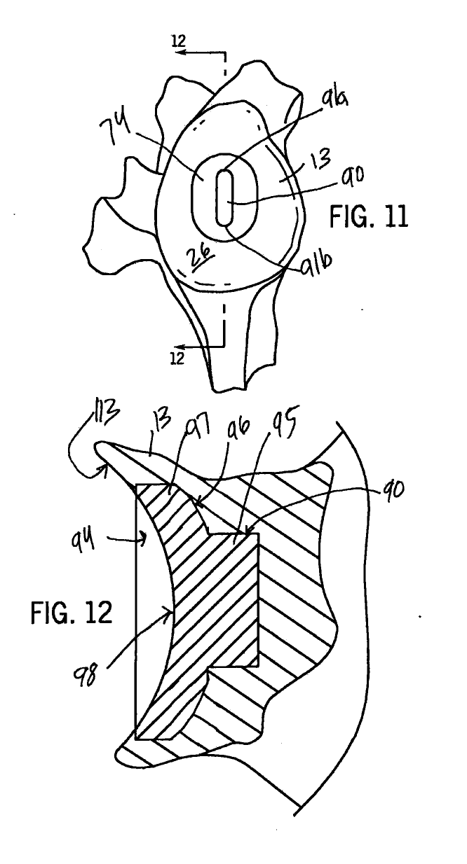

A second keel slot lengthening drill jig 80 is then used to lengthen the major axis of the slot 60 to accommodate a keeled prosthetic glenoid. Referring to FIGS. 10A and 10B, the second drill jig 80 has an oblong locating plug 81 extending from the bottom surface 82 which corresponds in shape to the slot 80 made in the glenoid 13 to set the proper angle for the keel slot lengthening drilling operation. A superior-inferior running line 83 on the second drill jig 80 is aligned with the vertical superior-inferior centerline 24 marked on the surface 26 of the glenoid 13.

Steadying the second drill jig 80 using the handle 84, the outer perimeter of the oblong plug 81 is inserted into the slot 60 in the glenoid 13 to set the superior-inferior location for two additional holes. Using the same drill bit 45 as in the previous two drilling operations, the drill-stop collar 44 on the drill bit 45 is set to a second position 47b in FIG. 4. This position will be used with the second drill jig 80. The second drill jig 80 sits inside of the oblong recess 74 so the depth setting on the drill bit 45 needs to be adjusted to compensate for this. The body 85 of the second drill jig 80 is inserted into the recess 74, lining-up the keel-locating plug 81 on the bottom of the second drill jig 80 with the slot 60 in the glenoid 13.

Without removing the second drill jig 80 from the recess 74, the drill bit 45 is inserted into a first guide bore 86 of the second drill jig 80 and pressure is applied until the drill-stop collar 44 touches the top surface 88 of the second drill jig 80 indicating that one hole is drilled to the proper depth. The drill bit 45 is then inserted into a second guide bore 87 of the second drill jig 80 and pressure is applied until the drill-stop collar 44 touches the top surface 88 of the second drill jig 80 indicating that another hole is drilled to the proper depth. The second drill jig 80 is then removed from the recess 74 in the glenoid 13. A chisel or scalpel is used as required to remove the bone between the first hole and the second hole, thereby making an oblong slot 90 with rounded ends 91a, 91b. The resulting slot 90 is shown in FIG. 11.

A suitable prosthetic glenoid component 94 includes an oblong keel 95 extending from a bottom surface 96 of the body 97 of the glenoid component 94. The glenoid component 94 can comprise stainless steel, cobalt chrome, titanium, or polyethylene and combinations thereof (e.g., metal backed polyethylene). The oblong keel 95 fits in the slot 90. Following the successful dry-fit of the keeled glenoid component 94 of the proper size, the glenoid component 94 can be cemented into place in the glenoid 13 as shown in FIG. 12 using bone cement such as polymethylmethacrylate. The glenoid component 94 has a generally spherical bearing surface 98 which is configured to line up with the non-resected preserved peripheral bearing surface 113 of the glenoid 13.

Thus, the invention provides a suite of custom tooling and a surgical technique for a process in which a prosthetic glenoid component is inset into the face of the existing glenoid bone. The methods and tools can also be used with robotic-assisted surgeries. One non-limiting example robotic system is the robotic arm interactive orthopedic system sold under the name RIO® by MAKO Surgical Corp., Ft. Lauderdale, Fla., USA. This robotic system can use a hand-held burr (as opposed to a reamer) to prepare the bone surface for implantation of the new prosthesis.

### INDUSTRIAL APPLICABILITY

This invention relates to a suite of custom tooling and a surgical technique for a process in which a prosthetic glenoid component is inset into the face of the existing glenoid bone.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A kit for use in shoulder arthroplasty, the kit comprising:
a glenoid component including a body having a first articulating surface and a second surface opposite the first articulating surface, the first articulating surface being suitable for engaging a head of a humerus or a humeral implant, the second surface being suitable for being secured to a scapula, the glenoid component further including a keel extending away from the second surface;
a first drill jig having a locating pin and a guide hole dimensioned to receive a drill bit; and
a cutting tool having a follower pin and a cutting surface, the cutting surface of the cutting tool being dimensioned to create a recess in the surface of the glenoid cavity of the scapula, the recess having a size such that a periphery of the glenoid bone is preserved when the recess is created in the surface of the glenoid cavity.

2. A kit for use in shoulder arthroplasty, the kit comprising:
a glenoid component including a body having a first articulating surface and a second surface opposite the first articulating surface, the first articulating surface being suitable for engaging a head of a humerus or a humeral implant, the second surface being suitable for being secured to a scapula, the glenoid component further including a keel extending away from the second surface;
a transparent template having a cut-out guide dimensioned to receive a bone marking tool;
a first drill jig having a locating pin and a guide hole dimensioned to receive a drill bit; and
a cutting tool having a follower pin and a cutting surface.

3. The kit of claim 2 wherein:
the template has an indication of a centerline of the template.

4. The kit of claim 2 wherein:
the template includes an outline of a footprint of the glenoid component.

5. The kit of claim 1 wherein:
the cutting tool includes a depth-stop collar for adjusting depth of cut of the cutting tool.

6. (canceled)

7. (canceled)

8. The kit of claim 1 further comprising:
a second drill jig having a locating plug, having a first guide bore dimensioned to receive a drill bit, and having a second guide bore dimensioned to receive the drill bit.

9. (canceled)

10. (canceled)

11. The kit of claim 1 further comprising:
a drill bit dimensioned to pass through the guide hole of the first drill jig.

12. The kit of claim 11 further comprising:
a drill stop for adjusting depth of cut of the drill bit.

13. (canceled)

14. The kit of claim 1 wherein:
the follower pin and the cutting surface of the cutting tool are concentric and integral.

15. (canceled)

16. The kit of claim 1 wherein:
an outer perimeter of the first drill jig has the same geometry as an outer edge of the glenoid component.

17. (canceled)

18. (canceled)

19. The kit of claim 2 wherein:
the cutting surface of the cutting tool is dimensioned to create a recess in the surface of the glenoid cavity, the recess having a size such that a periphery of the glenoid bone is preserved.

20. The kit of claim 19 wherein:
the body of the glenoid component is dimensioned such that the glenoid component is flush with native peripheral glenoid bone when the glenoid component is secured to the scapula.

21. A method for implanting a glenoid component during shoulder arthroplasty, the method comprising:
(a) placing a template against a surface of a glenoid cavity of a scapula;
(b) inserting a bone marking tool through a cut-out guide in the template;
(c) using the bone marking tool to create a bone mark on the surface of the glenoid cavity;
(d) aligning a drill with the bone mark and drilling a first pilot hole in the surface of the glenoid cavity;
(e) placing a locating pin of a first drill jig in the first pilot hole;
(f) drilling through a guide hole of the first drill jig to create a second pilot hole in the surface of the glenoid cavity;
(g) removing bone material between the first pilot hole and the second pilot hole to create a slot in the surface of the glenoid cavity between the first pilot hole and the second pilot hole; and
(h) positioning a keel of a glenoid component in the slot and securing the glenoid component in the scapula.

22. (canceled)

23. The method of claim 21 wherein:
the template is transparent.

24. The method of claim 21 wherein:
the template includes an outline of a footprint of the glenoid component.

25. The method of claim 21 wherein:
step (g) further comprises placing a follower pin of a cutting tool in the slot and moving the follower pin from a first position to a second position in the slot such that a cutting surface of the cutting tool removes bone material thereby creating a recess in the surface of the glenoid cavity surrounding the slot, and
step (h) further comprises positioning a body of the glenoid component in the recess.

26. The method of claim 25 wherein:
the cutting tool includes a depth-stop collar for adjusting depth of cut of the cutting tool.

27. (canceled)

28. (canceled)

29. (canceled)

30. The method of claim 25 wherein:
the cutting surface of the cutting tool has a first curvature approximately equal to a second curvature of a surface of the glenoid component that contacts the surface of the glenoid cavity when the glenoid component is secured in the scapula.

31. (canceled)

32. (canceled)

33. (canceled)

34. (canceled)

35. The method of claim 21 wherein:
step (h) comprises securing the glenoid component in the scapula such that a bearing surface of the glenoid component lines up with a non-resected section of the surface of the glenoid cavity.

36. (canceled)

37. (canceled)

38. The method of claim 21 wherein:
step (g) further comprises placing a follower pin of a cutting tool in the slot and moving the follower pin from a first position to a second position in the slot such that a cutting surface of the cutting tool removes bone material thereby creating a recess in the surface of the glenoid cavity surrounding the slot wherein the cutting surface of the cutting tool is dimensioned such that the recess created has a size such that a periphery of the glenoid bone is preserved, and
step (h) further comprises positioning a body of the glenoid component in the recess such that the glenoid component is flush with native peripheral glenoid bone.
